(19) United States
(12) Patent Application Publication
(10) Pub. No.: US 2017/0318835 A1
(43) Pub. Date: Nov. 9, 2017

(54) METHOD FOR SEPARATING ALPHA-LACTALBUMIN AND BETA-LACTOGLOBULIN

(71) Applicant: Upfront Chromatography A/S, Copenhagen Ø (DK)

(72) Inventors: Allan Otto Fog Lihme, Farum (DK); Marie Bendix Hansen, Frederiksberg (DK)

(73) Assignee: Upfront Chromatography A/S, Copenhagen Ø (DK)

(21) Appl. No.: 15/503,924

(22) PCT Filed: Aug. 14, 2015
(86) PCT No.: PCT/DK2015/000033

§ 371 Date: Feb. 14, 2017

(30) Foreign Application Priority Data
Aug. 15, 2014 (DK) PA 2014 00449
Aug. 15, 2014 (DK) PA 2014 00450
Aug. 21, 2014 (DK) PA 2014 00468

(51) Int. Cl. A23J 1/20; C07K 14/47; C07K 1/18; C07K 14/76; A23C 9/146

(52) U.S. Cl. CPC A23J 1/205; C07K 14/76; A23C 9/1465; C07K 1/18; C07K 14/4717

(57) ABSTRACT

The present invention relates to a method for providing an alpha-lactalbumin fraction and a beta-lactoglobulin fraction from a whey material obtained from milk, the method comprising the steps of: (i) providing the whey material; (ii) contacting the whey material with a chromatographic support allowing beta-lactoglobulin to be retained by the chromatographic support; (iii) obtaining a permeate fraction from the chromatographic support comprising the alpha-lactalbumin fraction; (iv) optionally washing the chromatographic support; and (v) obtaining a retentate fraction from the chromatographic support comprising the beta-lactoglobulin fraction; wherein the whey material provided in step (i) has been depleted, or substantially depleted from at least one whey protein, such as at least 2 whey proteins, e.g. at least 3 whey proteins.

### TECHNICAL FIELD OF THE INVENTION

The present invention relates to a method for separating the whey proteins alpha-lactalbumin and beta-lactoglobulin from a whey material obtained from milk. In particular, the present invention relates to the separation of alpha-lactalbumin and beta-lactoglobulin from a whey material from which at least one whey protein has been removed, or substantially removed.

### BACKGROUND OF THE INVENTION

Milk is a very complex material and industrial processes use milk to produce casein, whey, lactose, condensed milk, powdered milk, and many other food-additives and industrial products. Milk comprises a mixture of components, such as proteins, minerals, fat, sugars, salts, and vitamins. In particular, the proteins in milk, which are mainly found as casein proteins or whey proteins, have gained increasingly attention over the years. The reason for this increased interest lies in the diversity of milk proteins and because each protein has unique attributes to nutritional, biological, functional and food ingredient applications. Furthermore, these proteins constitute, together with e.g. peptides and enzymes in milk, a major and important health and nutritional role in humans and animals.

To achieve highest possible potential of proteins and to explore or exploit the potentially functional and bioactive properties of proteins, and whey proteins, it is important to isolate native whey proteins by a procedure that avoids possible denaturing conditions (such as high salt conditions, high or low pH conditions, heat or protease treatment/exposure).

Except from casein products, like cheese, the most commonly produced milk protein products are Whey Protein Concentrates (WPC) and Whey Protein Isolates (WPI). These WPC and WPI products are standard products obtained from whey through various separation techniques, such as precipitation techniques, membrane filtration techniques as well as ion exchange adsorption procedures. Further fractionation of the WPC proteins or the WPI proteins into individual protein fractions, such as a beta-lactoglobulin fraction, an alpha-lactalbumin fraction, an immunoglobulin fraction, a lactoperoxidase fraction, and a lactoferrin fraction, is made possible by using a chromatographic support.

Separation of the individual whey protein factions have proven to be difficult due to the relatively similar physicochemical properties of the different whey proteins. The skilled person knows that it is difficult to provide a good separation based on the molecular size of the whey proteins (as required when using membrane filtration) and the fractions provided in this way results in poor yields and/or poor purities because of the complexity of the whey material and the whey proteins to be isolated.

However, separating whey proteins based on their isoelectric point (pI) gives two distinct groups: the major proteins, like alpha-lactalbumin; beta-lactoglobulin, immunoglobulin G and serum albumin, which are negatively charged at the pH of sweet whey (pH 6.2-6.4); and the minor whey proteins, like lactoferrin and lactoperoxidase, that hold a positive net charge at the pH of sweet whey. These distinct properties offer the possibility of selectively separating one group from another, using a chromatographic support.

Such selective separation using chromatographic supports, in providing individual fractions like a protein fraction comprising beta-lactoglobulin and a fraction comprising alpha-lactalbumin, can be operated under two conditions:
(i) Selective elution of the whey proteins from the chromatographic support, or
(ii) Selective adsorption of one or more whey proteins to the chromatographic support

In selective elution all proteins in a solution are captured simultaneously onto the chromatographic support. The chromatographic support is rinsed from contaminants and un-captured matter. Then the proteins captured are sequentially eluted one by one using specially designed elution buffers suitable for the particular protein to be isolated. Thus, by using the selective elution technique it is possible to obtain several purified protein fractions from the same chromatographic support. In this manner, the production costs are spread among different products.

Some of the challenges with selective elution is that the loading capacity when loading the whey on to the column is low and the buffer consumption when eluting the various protein fractions is high. Furthermore, the protein fractions obtained have low yield, low recovery and/or a low purity because of overlapping elution conditions between the different protein fractions and a high salt content (conductivity).

In selective adsorption, the process conditions are optimised to capture one protein over another. When the protein of interest is captured, the chromatographic support may be rinsed from contaminants followed by elution of the specific protein.

The skilled person does not consider the selective adsorption technique to have great potential for developing into industrial applications, because the technique only provides optimal binding for a single protein. The revenue from this single protein must then cover all the production costs as well as costs in connection with disposal of the remaining whey proteins. Thus, the selective elution technique is considered most appropriate for industrial applications.

Furthermore, selective adsorption is rarely achievable in an industrial setting because it requires a unique and expensive adsorbent setting and precise adjustment of adsorption conditions.

Thus, the selective elution technique is considered most appropriate for industrial applications.

Hence, an improved method for providing whey protein fractions, which would solve the above issues and which would be suitable for industrial application, would be advantageous. In particular, a more efficient method resulting in increased yield, recovery and purity of alpha-lactalbumin and beta-lactoglobulin, having a low buffer consumption and a high loading capacity is desirable.

### SUMMARY OF THE INVENTION

Accordingly, it is an object of the present invention to provide an improved method for separating alpha-lactalbumin from beta-lactoglobulin. The method is cost effective and result in high quality protein fractions, providing an individual alpha-lactalbumin fraction and an individual beta-lactoglobulin fraction both having high purity, high recovery and/or high yield.

In particular, it is an object of the present invention to provide a method that solves the above mentioned problems of the prior art with separating alpha-lactalbumin from beta-lactoglobulin.

Thus, one aspect of the invention relates to a method for providing an alpha-lactalbumin fraction and a beta-lactoglobulin fraction from a whey material obtained from milk, the method comprising the steps of:
(i) Providing the whey material;
(ii) Contacting the whey material with a chromatographic support allowing beta-lactoglobulin to be retained by the chromatographic support;
(iii) Obtaining a permeate fraction from the chromatographic support comprising the alpha-lactalbumin fraction;
(iv) Optionally washing the chromatographic support;
(v) Obtaining a retentate fraction from the chromatographic support comprising the beta-lactoglobulin fraction;
   wherein the whey material provided in step (i) has been depleted, or substantially depleted from at least one whey protein, such as at least 2 whey proteins, e.g. at least 3 whey proteins.

Another aspect of the present invention relates to an alpha-lactalbumin fraction comprising alpha-lactalbumin and/or a beta-lactoglobulin fraction comprising beta-lactoglobulin obtainable by the method according to the present invention.

Yet another aspect of the present invention relates to using the alpha-lactalbumin fraction according to the present invention and/or the beta-lactoglobulin fraction according to the present invention, in a food product, a feed product, beverage product, a cosmetic product, a pharmaceutical product, or a food supplement.

The present invention will now be described in more detail in the following.

### DETAILED DESCRIPTION OF THE INVENTION

Over the years, the number of applications for which whey proteins are used have enlarged and the requirement for isolated and pure fractions have gained more and more interest. One of the challenges with the currently described methods, in addition to the above-mentioned disadvantages of the prior art, is the costs for providing the different isolated fractions. Hence, the inventors of the present invention surprisingly found a method for providing an alpha-lactalbumin fraction and a beta-lactoglobulin fraction with high yield, high recovery and high purity which method is easy, fast, and which is cost efficient.

Thus, one aspect of the present invention relates to a method for providing an alpha-lactalbumin fraction and a beta-lactoglobulin fraction from a whey material obtained from milk by selective adsorption of the beta-lactoglobulin fraction, the method comprising the steps of:
(i) Providing the whey material;
(ii) Optionally adjusting the pH of the whey material;
(iii) Contacting the whey material with a chromatographic support allowing the beta-lactoglobulin to be retained by the chromatographic support;
(iv) Obtaining a permeate fraction from the chromatographic support comprising the alpha-lactalbumin fraction
(v) Optionally washing the chromatographic support;
(vi) Contacting an elution buffer to the chromatographic material; and
(vii) Obtaining a retentate fraction from the chromatographic support comprising the beta-lactoglobulin fraction;
   wherein the chromatographic support comprise one or more negatively charged ligands capable of binding the beta-lactoglobulin fraction.

Another aspect of the present invention relates to a method for providing an alpha-lactalbumin fraction and a beta-lactoglobulin fraction from a whey material obtained from milk, the method comprising the steps of:
(i) Providing the whey material;
(ii) Contacting the whey material with a chromatographic support allowing beta-lactoglobulin to be retained by the chromatographic support;
(iii) Obtaining a permeate fraction from the chromatographic support comprising the alpha-lactalbumin fraction;
(iv) Optionally washing the chromatographic support;
(v) Obtaining a retentate fraction from the chromatographic support comprising the beta-lactoglobulin fraction;
   wherein the whey material provided in step (i) has been depleted, or substantially depleted from at least one whey protein, such as at least 2 whey proteins, e.g. at least 3 whey proteins.

In the present context the term “depleted” relates to no detectable amount of the given whey protein is present in the whey material.

In the present context the term “substantially depleted” relates to a whey material wherein the content of a given whey protein has been reduced, relative to an initial content of said whey protein in whey, to a content of less than 30%, of the initial content of said protein, such as less than 20%, e.g. less than 15%, such as less than 10%, e.g. less than 5%, such as less than 3%, e.g. less than 1%, such as less than 0.5%, e.g. less than 0.1%, such as less than 0.05%, e.g. less than 0.01%.

The “initial content” of the whey protein in whey obtained directly from removal of casein may be determined by:
(a) Analysis of the whey used without depletion (or without substantial depletion), or
(b) Traditional listed amount of said protein in whey as described in the literature.

In a preferred embodiment of the present invention the fractionation of the alpha-lactalbumin fraction from the beta-lactoglobulin fraction is performed by selective adsorption of the beta-lactoglobulin fraction to the chromatographic support.

In present context, the term “selective adsorption” relates to a process where the chromatographic support is designed and/or where the process conditions are designed to favour binding of one component rather than another component from a mixture. In respect of the present invention the “one component” would be beta-lactoglobulin and the “another component” would be alpha-lactglobulin, and “the mixture” would be whey material.

In an embodiment of the present invention the selective adsorption results in a separation of the alpha-lactalbumin fraction from the beta-lactoglobulin. This separation may be performed by providing a chromatographic support and/or process conditions, which favour selective adsorption of beta-lactoglobulin and allow alpha lactalbumin to pass the chromatographic support without being adsorbed.

In the present context, the term “retained” relates to the act of holding or keeping the beta-lactoglobulin in a particular place, namely in the chromatographic support. The beta-lactoglobulin may be retained in the chromatographic support until the conditions are changed and the beta-lactoglobulin is liberated and eluted from the chromatographic support.

In an embodiment of the present invention the method for providing the alpha-lactalbumin fraction and the beta-lactoglobulin fraction may be a medium size scale production or large scale production.

### Whey

According to the method described by the present invention the initial step relates to providing of a whey material. The whey material of the present invention is a fractional whey comprising alpha-lactalbumin and beta-lactoglobulin, wherein at least one whey protein has been depleted or substantially depleted.

In an embodiment of the present invention; the whey material may be obtained from any milk producing animal, and preferably animals traditionally used for large-scale milk production. Preferably, the milk is obtained from ruminant animals, such as cattle, goats, sheep, giraffes, yaks, deer, camels, llamas or antelope.

In the present context, the term “whey material” relates to the serum material from milk, the part of milk without casein. Whey material according to the present invention may be a fractionated whey obtained from whey, acidic whey, sweet whey, whey protein isolates (WPI) or whey protein concentrates (WPC).

In an embodiment of the present invention, the whey material comprises less than 5 g casein/L whey material, such as less than 2 g casein/L whey material, e.g. less than 1 g casein/L whey, such as less than 0.5 g casein/L whey, such as less than 0.2 g casein/L whey material, e.g. less than 0.1 g casein/L whey, such as less than 0.05 g casein/L whey material, e.g. less than 0.01 g casein/L whey.

The whey material according to the present invention as provided in step (i) has been depleted or substantially depleted in at least one whey protein, such as at least 2 whey proteins, e.g. at least 3 whey proteins.

In an embodiment of the present invention the at least one protein may be selected from the group consisting of immunoglobulin G, serum albumin, lactoferrin, lactoperoxidase and glycomacropeptide.

In a preferred embodiment of the present invention the at least one protein may be selected from the group consisting of immunoglobulin G, serum albumin and glycomacropeptide.

In yet an embodiment of the present invention the whey material comprises less than 30% (w/w) on a dry matter basis of at least one protein selected from the group consisting of immunoglobulin G, serum albumin, and glycomacropeptide relative to the total amount of protein in the whey material, more preferably, less than 20%, even more preferably, less than 15%, even more preferably, less than 10%, even more preferably, less than 5%, even more preferably, less than 2%, even more preferably, less than 1%, even more preferably, less than 0.5%, even more preferably, less than 0.1%, even more preferably, less than 0.05%.

In a preferred embodiment the whey material provided in step (i) may be depleted or substantially depleted in immunoglobulin G. Preferably, the whey material may comprise less than 8% (w/w) on a dry matter basis of immunoglobulin G relative to the total amount of protein in the whey material, more preferably, less than 5%, even more preferably, less than 3%, even more preferably, less than 2%, even more preferably, less than 1%, even more preferably, less than 0.5%, even more preferably, less than 0.1%, even more preferably, less than 0.05%.

In yet a preferred embodiment of the present invention the whey material provided in step (i) may be depleted or substantially depleted in serum albumin. Preferably, the whey material may comprise less than 5% (w/w) on a dry matter basis of serum albumin relative to the total amount of protein in the whey material, more preferably, less than 4%, even more preferably, less than 3%, even more preferably, less than 2%, even more preferably, less than 1%, even more preferably, less than 0.5%, even more preferably, less than 0.1%, even more preferably, less than 0.05%.

In the event casein is removed by precipitation or curdling using rennet, glycomacropeptides (GMP) may be produced which will remain in the whey (as a “whey protein”) and a sweet whey is provided. In the present context depletion or substantial depletion of glycomacropeptides (GMP) only applies when the whey material is provided from sweet whey.

In a further preferred embodiment of the present invention the whey material provided in step (i) has been depleted or substantially depleted in glycomacropeptide. Preferably, the whey material may comprise less than 20% (w/w) on a dry matter basis of glycomacropeptide relative to the total amount of protein in the whey material, more preferably, less than 15%, even more preferably, less than 10%, even more preferably, less than 5%, even more preferably, less than 3%, even more preferably, less than 2%, even more preferably, less than 1%, even more preferably, less than 0.5%, even more preferably, less than 0.1%, even more preferably, less than 0.05%.

In an embodiment of the present invention, the method for providing the alpha-lactalbumin fraction and the beta-lactoglobulin fraction may be a batch process or a continuous process.

Medium size scale production and/or industrial scale production may be performed in a batch process. Preferably such batch process involves processing at least 50 litres whey per cycle, such as at least 100 litres whey per cycle, e.g. 250 litres whey per cycle, such as at least 500 litres whey per cycle, e.g. 750 litres whey per cycle, such as at least 1,000 litres whey per cycle, e.g. 2,500 litres whey per cycle, such as at least 5,000 litres whey per cycle, e.g. 7,500 litres whey per cycle, such as at least 10,000 litres whey per cycle, e.g. 25,000 litres whey per cycle, such as at least 50,000 litres whey per cycle, e.g. 75,000 litres whey per cycle, such as at least 100,000 litres whey per cycle, e.g. 250,000 litres whey per cycle.

Alternatively, large scale production (industrial scale production) may be conducted at a continuous process. In selective separation, like selective adsorption, the process will eventually require an elution of the adsorbed protein, e.g. beta-lactoglobulin. By providing at least two chromatographic supports and placing them in parallel, such continuous selective adsorption process may be provided where the flow of whey material may be shifted from one chromatographic support, when this chromatographic material is loaded and ready for elution, to the other chromatographic support. Alternatively, moving bed chromatography, simulated moving bed chromatography or the like may be used.

In an embodiment of the present invention, the continuous selective adsorption process may have a capacity of at least 5,000 litres whey material per hour, such as at least 10,000 litres whey material per hour, e.g. at least 12,000 litres whey per hour, such as at least 15,000 litres whey material per hour, e.g. at least 18,000 litres whey per hour, such as at least 20,000 litres whey material per hour, e.g. at least 25,000 litres whey per hour, such as at least 50,000 litres whey material per hour, e.g. at least 100,000 litres whey per hour.

In order to achieve the desired separation of beta-lactoglobulin, favourable process conditions may be provided. Thus, the whey material may have a conductivity at 20° C. of at least 3.0 mS/cm, such as at least 3.5 mS/cm, e.g. at least 4.0 mS/cm, such as at least 4.5 mS/cm, e.g. at least 5 mS/cm, such as at least 5.5 mS/cm, e.g. at least 6, such as at least 7 mS/cm, e.g. at least 7.5 mS/cm, such as in the range of 4.5-15 mS/cm, such as in the range of 5.0-14 mS/cm, e.g. in the range of 5.5-13 mS/cm, such as in the range of 6.0-12.5 mS/cm, e.g. in the range of 6.5-12 mS/cm, such as in the range of 7.0-11.5 mS/cm, e.g. in the range of 7.5-11 mS/cm, such as in the range of 8.0-10.5 mS/cm, e.g. about 9.0 mS/cm. Preferably, the conductivity of the whey material is not adjusted.

In another embodiment of the present invention, the whey material may have a conductivity at 20° C. of less than 7 mS/cm, such as less than 5, e.g. less than 3, and a pH-value in the range of 4.6-6.5, such as a pH-value in the range of 4.7-6.4, e.g. a pH-value in the range of 4.8-6.3, such as a pH-value in the range of 4.9-6.2, e.g. a pH-value in the range of 4.9-6.1, such as a pH-value in the range of 5.0-6.0, e.g. a pH-value in the range of 5.2-5.8,

In an embodiment of the present invention, the whey material may comprise minerals. In a preferred embodiment of the present invention the whey material has not been subjected to removal of minerals. In particular the whey material has not been subjected to removal of calcium.

Preferably, the mineral is selected from the group consisting of calcium, phosphorus, iodine, magnesium, zinc, and potassium. Preferably, the mineral(s) present in the whey material is/are naturally present in the whey material.

In the present context the, term “naturally present” relates to the minerals present in the whey material and are not a separately added compound, but found in the whey material provided in step (i).

### pH Adjustment

Depending on the type of the whey material for providing the alpha-lactalbumin fraction and the beta lactoglobulin fraction, the whey material may be subjected to an adjustment of the pH.

In a preferred embodiment of the present invention, the pH of the whey material may be adjusted in order to facilitate optimal adsorption of beta-lactoglobulin to the chromatographic support. In a preferred embodiment of the present invention the pH of the whey material is adjusted in step (ii) to a pH above 4.5, such a pH above 4.6, e.g. a pH above 4.7, such a pH above 4.8, e.g. a pH above 4.9, such a pH above 5.0, e.g. a pH above 5.1, such a pH above 5.2, e.g. a pH above 5.3, such a pH above 5.4, e.g. a pH above 5.5, such as in the range of pH 4.5-6.5, such as in the range of pH 4.5-6.0, e.g. in the range of pH 4.6-5.5, such as in the range of pH 4.7-5.0.

If pH of the whey material becomes too low, below 4.0 there is an increased risk that the proteins or part of the proteins, may denature and the native functionality may be lost.

Adjusting the pH is preferably done by adding an acid and lowering the pH. Preferably, low cost mineral acids such as hydrochloric acid, phosphoric acid, sulphuric acid may be used. However, food grade organic acids such as acetic, citric and lactic acid may also be particularly preferred.

Alternatively, the pH value of the whey material may be adjusted by passing the whey material through a strong cation exchanger (acidic form). The cation exchanger will bind salts from the whey material and release H⁺-ions and thereby decrease pH to the desired value. Cation exchangers and process suitable for lowering the pH value are well known to the skilled person.

In an embodiment of the present invention the whey material is loaded on to the chromatographic support at a flow-rate in the range of 1-50 cm/min; preferably in the range of 5-30 cm/min; more in the range of 10-25 cm/min; even more preferably, in the range of 15-20 cm/min.

### Chromatographic Support

As described above, the present invention teach the use of a chromatographic support allowing beta-lactoglobulin to be retained, step (v).

In the present context, the term “chromatography support” relates to any kind of container comprising an adsorbent, which can be supplied with at least one inlet for the application of the whey material and at least one outlet for obtaining the alpha-lactalbumin fraction and/or the beta-lactoglobulin fraction when subjected to an elution buffer.

The chromatographic support to be used may be a membrane chromatography support, preferably a charged membrane chromatography support, or a column chromatography support. Preferably, the column chromatography support includes a Packed Bed Chromatography, stirred tank adsorption, moving bed chromatography, simulated moving bed chromatography, Fluidized Bed Chromatography and/or Expanded Bed Chromatography.

The fact that the Expanded Bed Chromatography (EBA) technology generally can work efficiently with non-clarified raw materials makes it an attractive solution to implement for the isolation and fractionation of biomolecular substances, such as alpha-lactalbumin and beta-lactoglobulin from whey material. Compared to processes based on packed bed chromatography, Expanded Bed Chromatography may offer a robust process comprising fewer steps and thus results in increased yields and an improved process economy. Due to the expansion of the adsorbent bed during execution of an EBA process, EBA columns may further be scaled up to industrial scale without any significant considerations regarding increased back pressures or breakdown of the process due to clogging of the system which is often a problem when using packed bed columns. Hence, Expanded Bed Chromatography may be the preferred column chromatographic support according to the present invention.

Generally, the Expanded Bed Adsorption is well known to the person skilled in the art, and the method described in the present invention may be adapted to the processes described in WO 92/00799, WO 92/18237, WO 97/17132, WO 00/57982 or WO 98/33572.

### Adsorbent

In a preferred embodiment of the present invention, the chromatographic support may comprise an adsorbent.

This adsorbent may be used in a technique selected from the group consisting of ion exchange adsorption, hydrophobic interaction adsorption, affinity adsorption, mixed mode ligand adsorption, metal chelate adsorption, reversed phase adsorption, and any combination hereof.

In a preferred embodiment of the present invention, the adsorbent may be used in ion exchange adsorption, preferably, in cation exchange adsorption.

Before the whey material may be contacted with the adsorbent an initial, but optional, step in the method of the invention may involves equilibration of the adsorbent. Such equilibration may be done by using an equilibration liquid.

In a preferred embodiment, the equilibration liquid may be used in cation exchange adsorption and be an aqueous liquid having a pH above 4.5, such a pH above 4.6, e.g. a pH above 4.7, such a pH above 4.8, e.g. a pH above 4.9, such a pH above 5.0, e.g. a pH above 5.1, such a pH above 5.2, e.g. a pH above 5.3, such a pH above 5.4, e.g. a pH above 5.5, such as in the range of pH 4.5-6.5, such as in the range of pH 4.5-6.0, e.g. in the range of pH 4.6-5.5, such as in the range of pH 4.7-5.0.

Equilibration of the adsorbent may preferably be done by using an acid. Preferably, the equilibration liquid used in cation exchange adsorption may comprise low cost mineral acids such as hydrochloric acid, phosphoric acid, sulphuric acid. However, food grade organic acids such as acetic, citric and lactic acid may also be particularly preferred.

In a preferred embodiment of the present invention, the adsorbent may be used in ion exchange adsorption, preferably, in anion exchange adsorption.

In a preferred embodiment, the equilibration liquid may be used in anion exchange adsorption and be an aqueous liquid having a pH above 6.5, such a pH above 7.0, e.g. a pH above 7.5, such a pH above 8.0, e.g. a pH above 8.5, such a pH above 9.0, such as in the range of pH 7.0-9.0.

In an embodiment of the present invention the equilibration liquid may be used in anion exchange adsorption may comprise sodium hydroxide, potassium hydroxide, calcium hydroxide, ammonium hydroxide, potassium phosphate, sodium phosphate, sodium citrate, sodium acetate, sodium carbonate or any combinations hereof. Preferably, the elution buffer comprises sodium hydroxide, potassium hydroxide, calcium hydroxide, ammonium hydroxide or any combination hereof is preferred.

In the present context the term “adsorbent” relates to the entire bed present in the chromatographic support and is responsible for retaining the beta-lactoglobulin. In an embodiment of the present invention the adsorbent may comprise individual particles. In the present context the term “adsorbent particle” is used interchangeably with the term “particle” and relates to the individual single particles which makes up the adsorbent.

In another embodiment of the present invention the adsorbent may comprise a membrane charged with a negatively charged ligand capable of binding the beta-lactoglobulin fraction.

In the event the adsorbent is used in Expanded bed Adsorption several features, such as the flow rate, the size of the particles and the density of the particles all have influence on the expansion of the fluid bed and the separation of the proteins. It is important to control the degree of expansion in such a way to keep the adsorbent particles inside the column, but at the same time optimize the flow rate.

The degree of expansion may be determined as H/HO, where “HO” is the height of the bed in packed bed mode and “H” is the height of the bed in expanded mode. In an embodiment of the present invention the degree of expansion H/HO is in the range of 1.0-10 e.g. 1.0-6, such as 1.2-5, e.g. 1.3-5, such as 1.5-4, e.g. 4-6, such as 3-5, e.g. 3-4, such as 4-6.

In another embodiment of the present invention the degree of expansion H/HO is at least 1.0, such as at least 1.5, e.g. at least 2, such as at least 2.5, e.g. at least 3, such as at least 3.5, e.g. at least 4, such as at least 4.5, e.g. at least 5, such as at least 5.5, e.g. at least 6, such as at least 10.

Furthermore, the density of the EBA adsorbent particle is found to be highly significant for the applicable flow rates in relation to the maximal degree of expansion of the adsorbent bed possible inside a typical EBA column (e.g. H/HO max 3-5) and must be at least 1.3 g/ml, more preferably at least 1.5 g/ml, still more preferably at least 1.8 g/ml, even more preferably at least 2.0 g/ml, most preferably at least 2.3 g/ml, in order to enable a high productivity of the method.

The density of the EBA adsorbent particle is meant to be the density of the adsorbent particle in it's fully solvated (e.g. hydrated) state as opposed to the density of a dried adsorbent particle.

In an embodiment of the present invention the adsorbent particle has a mean particle size of at most 250 μm, such as at most, 200 μm, e.g. at most 180 μm, particularly such as at most 160 μm, e.g. at most 150 μm, such as at most 140 μm, e.g. at most 130 μm, such as at most 120 μm, e.g. at most 110 μm, such as at most 100 μm, even more typically, the adsorbent particle has a mean particle size in the range of 90-250 μm, e.g. 100-200 μm, such as 120-180 μm, e.g. 140-160 μm.

It is to be understood that mean particle sizes below 100 μm such as below, 90 μm, e.g. below 80 μm, such as below 70 μm, e.g. below 60 μm, such as below 50 μm, e.g. below 40 μm, such as below 30 μm, e.g. below 20 μm, such as below 10 μm are also covered by the present invention. Using adsorbent particles having a mean particle size below 100 μm, however, leads to lower productivity compared to using adsorbent particles having a mean particle size at or above 100 μm.

The high density of the adsorbent particle may be, to a great extent, achieved by inclusion of a certain proportion of a dense non-porous core materials, preferably having a density of at least 4.0 g/ml, such as at least 10 g/ml, e.g. at least 16 g/ml, such as at least 25 g/ml. Typically, the non-porous core material has a density in the range of about 4.0-25 g/ml, such as about 4.0-20 g/ml, e.g. about 4.0-16 g/ml, such as 12-19 g/ml, e.g. 14-18 g/ml, such as about 6.0-15.0 g/ml, e.g. about 6.0-16 g/ml.

The adsorbent particle used according to the present invention may be at least partly permeable to the proteins present in the whey material in order to ensure a significant binding capacity in contrast to impermeable particles that can only bind the target molecule on its surface resulting in relatively low binding capacity. The adsorbent particle may be of an array of different structures, compositions and shapes.

The adsorbent particles may be constituted by a number of chemically derivatised porous materials having the necessary density and binding capacity to operate at the given flow rates per se. The particles may be either of the conglomerate type, as described in WO 92/00799, having at least two non-porous cores surrounded by a porous polymeric base matrix, or of the pellicular type having a single non-porous core surrounded by a porous polymeric base matrix.

In the present context the term “conglomerate type” relates to a particle of a particulate material, which comprises high density non-porous core beads, having a core material of different types and sizes, held together by porous polymeric base matrix, e.g. a core particle consisting of two or more high density particles held together by surrounding agarose (porous polymeric base matrix).

In the present context the term “pellicular type” relates to a composite of particles, wherein each particle consists of only one high density core material coated with a layer of porous polymeric base matrix, e.g. a high density stainless steel bead coated with agarose.

Accordingly, the term “at least one high density non-porous core” relates to either a pellicular core, comprising a single high density non-porous particle or it relates to a conglomerate core comprising more than one high density non-porous particle.

In the present context the term “core” relates to the core particles present inside the adsorbent. The core particle or core particles may be incidentally distributed within the porous polymeric base matrix and is not limited to be located in the centre of the adsorbent.

The non-porous core constitutes typically of at most 50% of the total volume of the adsorbent, such as at most 40%, e.g. at most 30%, such as at the most 25%, e.g. at the most 20%, such as at the most 10%, e.g. at the most 5%.

The person skilled in the art knows various non-porous core materials and various porous polymeric base matrix. Examples of non-porous core materials and porous polymeric base matrixes may be found in WO 2010/037736. The skilled person also knows methods of preparing the adsorbent according to the present invention, such methods of preparing the adsorbent may be described in WO 2010/03776, EP 0 538 350 or WO 97/17132.

During operation, the whey material may be contacted with the adsorbent and beta-lactoglobulin may be adsorbed or fixated to the adsorbent, whereas the alpha-lactalbumin fraction does not bind to the chromatographic support and run through the adsorbent. This adsorption may be performed under pressure. Particulate material and soluble impurities are optionally removed from the column during an optional washing.

When contacting the whey material with the adsorbent, the ratio between the adsorbent and the whey material may be optimized in order to provide a high capacity of the adsorbent and to obtain a high purity, high yield and/or high recovery of the alpha-lactalbumin fraction and the beta-lactoglobulin fraction to be isolated.

Thus, in an embodiment of the present invention the loading ratio of beta-lactoglobulin relative to the adsorbent is at least 2 mg beta-lactoglobulin loaded per ml adsorbent, such as at least 5 mg, e.g. at least 10 mg, such as at least 12 mg, e.g. at least 15 mg, such as at least 20 mg, e.g. at least 25 mg, such as at least 30 mg, e.g. at least 35 mg, such as at least 40 mg, e.g. at least 50 mg, such as at least 75 mg, e.g. at least 100 mg, such as at least 125 mg, e.g. at least 150 mg.

In another embodiment of the present invention the loading ratio of the whey relative to the adsorbent is at least 10 mg protein loaded per ml adsorbent, such as at least 12 mg, e.g. at least 15 mg, such as at least 20 mg, e.g. at least 25 mg, such as at least 30 mg, e.g. at least 35 mg, such as at least 50 mg, e.g. at least 75 mg, such as at least 100 mg, e.g. at least 150 mg, such as at least 175 mg, e.g. at least 200 mg.

### Ligand

In order for the adsorbent to act in selective adsorption of beta-lactoglobulin from alpha-lactalbumin the adsorbent may comprise a ligand.

In a preferred embodiment of the present invention the adsorbent may comprise one or more ligands having affinity for beta-lactoglobulin.

In the present context the term “ligand” relates to a compound covalently attached to the adsorbent and which possesses the adsorbing function of beta-lactoglobulin.

The ligand may be a low molecular weight compound and in an embodiment of the present invention the ligand may have a molecular weight of at the most 500 Dalton, such as at the most 250 Dalton, e.g. at the most 100 Dalton, e.g. at the most 50 Dalton.

In a preferred embodiment of the present invention the ligand may be negatively charged at pH 6.5 or below, such as at pH 6.0 or below, e.g. at pH 5.5 or below.

In a further embodiment of the present invention the negatively charged ligand is selected from the group consisting of sulfonic acid ligand(s), such as propane sulphonic acid or butane sulphonic acid, and/or carboxylic acid ligand(s), such as chloroacetic acid.

In an embodiment of the present invention the ligand may be positively charged at pH above 6.5, such a pH above 7.0, e.g. a pH above 7.5, such a pH above 8.0, e.g. a pH above 8.5, such a pH above 9.0, such as in the range of pH 7.0-9.0.

In yet an embodiment of the present invention the positively charged ligand is selected from the group consisting of Q-anion exchange ligand(s), such as quaternary ammonium anion, or DEAE-anion exchange ligand(s), such as diethylaminoethyl.

In order to improve capacity, purity and recovery the ligand concentration may also be important. Hence, in a preferred embodiment of the present invention the ligand concentration is in the range of 30-300 μmoles per ml sedimented adsorbent, e.g. 50-200 μmoles per ml sedimented adsorbent, such as 75-175 μmoles per ml sedimented adsorbent, e.g. 100-160 μmoles per ml sedimented absorbent, such as 120-145 μmoles per sedimented adsorbent.

In the present context the terms “sedimented adsorbent” or “adsorbent particle” means an adsorbent in it's fully solvated (e.g. hydrated) state as opposed to the density of a dried adsorbent.

### Wash

After the whey material has been contacted with the chromatographic support and the beta-lactoglobulin fraction has been allowed to bind to the adsorbent the method according to the present invention may also involve an optional step of washing using a wash buffer. Hence, the method for providing the alpha-lactalbumin fraction and the beta-lactoglobulin fraction may further comprise the step of:
(iv) optionally washing the chromatographic support

The step of washing the chromatographic support may be performed by using a wash buffer, whereby a wash fraction may be obtained.

Once the whey material have been contacted with the chromatographic support, the chromatographic support may be washed using a wash buffer having a pH value as outlined previously for the optimal adsorption of beta-lactoglobulin fraction to the cation exchange adsorption or the anion exchange adsorption. Preferably, the wash buffer has a pH value of 6.5 or below, such as pH 6.0 or below, e.g. pH 5.5 or below, such as pH 5.0 or below, e.g. pH 4.7 or below, such as pH 4.6 or below.

In a preferred embodiment of the present invention, the acids applicable for adjusting the pH value of the wash buffer may be selected from the group of acids outlined previously for adjusting the pH value of the whey material.

In an embodiment of the present invention the flow rate used for the washing step may be selected from the ranges outlined previously for loading the whey material to the chromatographic support.

### The Alpha-Lactalbumin Fraction

The inventors of the present invention surprisingly found a method where the whey material is contacted with the chromatographic support allowing beta-lactoglobulin to be retained by the chromatographic support and wherein a permeate fraction is obtained from the chromatographic support comprising the alpha-lactalbumin. In this way a beta-lactoglobulin fraction and an alpha-lactalbumin fraction could be provided in high yields, high recoveries and/or high purities by a simple, inexpensive and easy way.

In the present context, the term “permeate” relates to the fraction running through the chromatographic support when the whey material is contacted with the chromatographic support and the beta-lactoglobulin is retained.

In an embodiment of the present invention the run-through fraction obtained comprising the alpha-lactalbumin fraction may, in addition to alpha-lactalbumin comprise one or more components selected from the group consisting of, carbohydrate, fat, salt, peptide and traces of other proteins present in the whey material. If the whey material is a sweet whey material, the alpha-lactalbumin fraction may comprise glycomacropeptide (GMP), unless the whey material (the sweet whey) has been depleted or substantially depleted from GMP. If the chromatographic support is overloaded some of the beta-lactoglobulin fraction may also be present in the run-through fraction and “contaminate” the alpha-lactalbumin fraction.

In an embodiment of the present invention the alpha-lactalbumin fraction obtained in step (iv) has a conductivity at 20° C. of at least 3.0 mS/cm, such as at least 3.5 mS/cm, e.g. at least 4.0 mS/cm, such as at least 4.5 mS/cm, e.g. at least 5 mS/cm, such as at least 5.5 mS/cm, e.g. at least 6, such as at least 7 mS/cm, e.g. at least 7.5 mS/cm, such as in the range of 4.5-15 mS/cm, such as in the range of 5.0-14 mS/cm, e.g. in the range of 5.5-13 mS/cm, such as in the range of 6.0-12.5 mS/cm, e.g. in the range of 6.5-12 mS/cm, such as in the range of 7.0-11.5 mS/cm, e.g. in the range of 7.5-11 mS/cm, such as in the range of 8.0-10.5 mS/cm, e.g. about 9.0 mS/cm. Preferably, the conductivity is determined directly on the alpha-lactalbumin fraction obtained from step (iv).

It is desirable to obtain an alpha-lactalbumin fraction having a high purity of alpha-lactalbumin. Hence, the amount of alpha-lactalbumin in the alpha-lactalbumin fraction may be a least 25% relative to the total amount of protein in the alpha-lactalbumin fraction, such as at least 30%, e.g. 40%, such as at least 50%, e.g. 60%, such as at least 70%, e.g. at least 80%.

As mentioned previously, the alpha-lactalbumin fraction according to the present invention is not retained by the chromatographic support, but runs through the chromatographic support. In a preferred embodiment of the present invention the alpha-lactalbumin fraction and the run through fraction are the same.

If a wash buffer is used to remove un-adsorbed components to obtain a wash fraction, such wash fraction may be mixed with the alpha-lactalbumin fraction/run through fraction in order to improve the recovery of alpha-lactalbumin from the whey material.

In an embodiment of the present invention the alpha-lactalbumin fraction has a pH value substantially similar to the pH value of the whey material loaded on to the chromatographic support. Preferably, the alpha-lactalbumin fraction comprises a pH value in the range of 4.5-6.5, such as 4.6-6.0, e.g. 4.7-5.5, such as 4.8-5.2, e.g. 4.9-5.1.

Since the alpha-lactalbumin fraction may be similar to the run through fraction as mentioned above the presence of other components in the alpha-lactalbumin fraction may depend on the type of whey material contacted with the chromatographic material.

In an embodiment of the present invention, the alpha-lactalbumin fraction further comprises lactose, vitamins and/or minerals.

In another embodiment of the present invention, the alpha-lactalbumin fraction further comprises minerals. Preferably, the mineral may be selected from the group consisting of calcium, phosphorus, iodine, magnesium, zinc and potassium. Even more preferably, the mineral may be calcium and a second mineral selected from the group consisting of phosphorus, iodine, magnesium, zinc and potassium.

The minerals in the alpha-lactalbumin fraction may be mineral(s) from the whey material. Preferably, 100% of the minerals in the alpha-lactalbumin fraction comes from the whey material, e.g. such as at least 98% of the minerals in the alpha-lactalbumin fraction comes from the whey material, such as at least 95% of the minerals in the alpha-lactalbumin fraction comes from the whey material, e.g. at least 92% of the minerals in the alpha-lactalbumin fraction comes from the whey material, such as at least 90% of the minerals in the alpha-lactalbumin fraction comes from the whey material, e.g. at least 75% of the minerals in the alpha-lactalbumin fraction comes from the whey material, such as at least 50% of the minerals in the alpha-lactalbumin fraction comes from the whey material.

In an embodiment of the present invention at least 20% (w/w) of the minerals present in the whey material relative to the total amount of minerals in the whey material are present in the alpha-lactalbumin fraction, such as at least 30%, e.g. at least 40%, such as at least 50% e.g. at least 70%, such as at least 80% e.g. at least 90%, such as at least 95%, e.g. at least 98%, such as at least 99% e.g. at least 99.5%, such as at least 99.9%.

Due to the allergenic effect of beta-lactoglobulin there is an interest in the industry to limit the amount of beta-lactoglobulin in the alpha-lactalbumin fraction due to the various uses of alpha-lactalbumin for e.g. an ingredient for food and infant formulas.

In a further embodiment of the present invention the alpha-lactalbumin fraction comprises less than 20% non-alpha-lactalbumin proteins relative to the total amount of protein in the alpha-lactalbumin fraction, more preferably, less than 10%, even more preferably, less than 5%, even more preferably, less than 3%, even more preferably, less than 2%, even more preferably, less than 1%, even more preferably, less than 0.5%, even more preferably, less than 0.1%, even more preferably, less than 0.05%.

In yet an embodiment of the present invention the non-alpha-lactalbumin proteins comprises one or more proteins selected from the group consisting of beta-lactoglobulin, immunoglobulin G, serum albumin, lactoferrin, lactoperoxidase and GMP.

In a further embodiment of the present invention the alpha-lactalbumin fraction comprises less than 20% beta-lactoglobulin relative to the total amount of protein in the alpha-lactalbumin fraction, more preferably, less than 10%, even more preferably, less than 5%, even more preferably, less than 3%, even more preferably, less than 2%, even more preferably, less than 1%, even more preferably, less than 0.5%, even more preferably, less than 0.1%, even more preferably, less than 0.05%.

The whey material may comprise one or more other proteins such as immunoglobulin G, serum albumin, lactoferrin, lactoperoxidase and/or glycomacropeptide (GMP), and the type of the whey material contacted with the chromatographic support may influence the composition of the alpha-lactalbumin fraction. Preferably, only a small portion of immunoglobulin G, serum albumin, lactoferrin, and/or lactoperoxidase may be found in the alpha-lactalbumin fraction. Preferably, insignificant amounts of immunoglobulin G, serum albumin, lactoferrin, and/or lactoperoxidase may be found in the alpha-lactalbumin fraction.

In an embodiment of the present invention the alpha-lactalbumin fraction comprises less than 5% immunoglobulin G relative to the total amount of protein in the alpha-lactalbumin fraction, more preferably, less than 4%, even more preferably, less than 3%, even more preferably, less than 2%, even more preferably, less than 1%, even more preferably, less than 0.5%, even more preferably, less than 0.1%, even more preferably, less than 0.05%.

In yet an embodiment of the present invention the alpha-lactalbumin fraction comprises less than 15% glycomacropeptide relative to the total amount of protein in the alpha-lactalbumin fraction, such as less than 10%, e.g. less than 5%, such as less than 2%, e.g. less than 1%, such as less than 0.5%, e.g. less than 0.1%, such as less than 0.05%.

In a preferred embodiment of the present invention process conditions may be provided where, contrary to immunoglobulin G, serum albumin, lactoferrin, and/or lactoperoxidase, 0.20 glycomacropeptide (GMP) may not be retained by the chromatographic support, but may follow the run through fraction into the alpha-lactalbumin fraction. In such embodiment the alpha-lactalbumin fraction may comprises at least 5% glycomacropeptide relative to the total amount of protein in the alpha-lactalbumin fraction, such as at least 7%, e.g. at least 10%, such as at least 15%, e.g. at least 20%, such as at least 25%, e.g. at least 30%, such as at least 40%.

In an embodiment of the present invention, the alpha-lactalbumin fraction obtained may be subjected to a first concentration step. Such first concentration step may include ultrafiltration, nanofiltration, microfiltration, centrifugation or any combination hereof. The first concentration step may result in a first concentrated retentate fraction comprising the alpha.lactalbumin fraction and a first concentrated permeate fraction comprising water, lactose and minerals. In an embodiment of the present invention the first concentrated permeate fraction may be subjected nanofiltration and/or microfiltration providing a water permeate, which may preferably be reused in the method of the present invention, and a lactose-retentate comprising lactose and minerals.

In an embodiment of the present invention, the alpha-lactalbumin fraction is a liquid, a concentrate or a powder.

### Elution of the Beta-Lactoglobulin

In order to obtain the retentate fraction from the chromatographic support comprising the beta-lactoglobulin fraction, the chromatographic support may be subjected to an elution buffer.

In the context of the present invention the term “elution buffer” relates to a composition capable of changing the conditions of the chromatographic support from specific adsorption of beta-lactoglobulin to the release and elution of the beta-lactoglobulin fraction.

In a preferred embodiment of the present invention the amount of elution buffer used for providing the beta-lactoglobulin fraction correspond to at the most 5 times the volume of the chromatographic support, such as at most 4 times the volume of the chromatographic support, e.g. at most 3 times the volume of the chromatographic support, such as at most 2 times the volume of the chromatographic support, e.g. at most 1 times the volume of the chromatographic support.

The inventors of the present invention surprisingly found, contrary to the expectations in the industry and in the prior art, that by providing the method of selective adsorption as described in the present invention, the cost of manufacturing or isolating a specific whey protein fraction was significantly lower than whey protein fractions isolated using the traditionally used specific elution, and at the same time higher yield and higher purity may be obtained.

If it is assumed that 3 column volumes are necessary for providing sufficient elution of a desired whey protein fraction (e.g. the alpha-lactalbumin fraction or the beta-lactoglobulin fraction) from the chromatographic support.

By using selective elution to fractionate the whey material 1 column volume adsorbent may be used to capture 1 kg alpha-lactalbumin and 1 kg beta-lactoglobulin. In order to obtain the protein fractions, 3 column volumes are used to elute the alpha-lactalbumin fraction and 3 column volumes are used to elute the beta-lactoglobulin fraction resulting in a total consumption of elution buffer of 6 column volumes.

By using selective adsorption to separate e.g. beta-lactoglobulin fraction and alpha-lactalbumin fraction two chromatographic supports may be provided, one for each protein fraction. Each of said two chromatographic supports comprises ½ column volume adsorbent which may be used to capture 1 kg alpha-lactalbumin fraction and 1 kg beta-lactoglobulin fraction, respectively. In order to obtain the protein fractions, 1.5 column volumes (3×½ column volumes) are used to elute the alpha-lactalbumin fraction and 1.5 column volumes (3×½ column volumes) are used to elute the beta-lactoglobulin fraction resulting in a total consumption of elution buffer of 3 column volumes.

Hence, by using the selective adsorption as described in the present invention it is possible to reduce the elution buffer solution by 50%.

In the present invention the consumption of elution buffer may be even further reduced since the alpha-lactalbumin fraction may be found in the permeate fraction obtained directly from the chromatographic support and therefore there is no need for any elution buffer for obtaining the alpha-lactalbumin fraction. Thus, the consumption of elution buffer for obtaining the alpha-lactalbumin fraction and the beta-lactoglobulin fraction may be reduced even further, preferably about 50% reduced. Thus, this may lead to a total reduction of the elution buffer consumption of about 75%.

In a preferred embodiment of the present invention the amount of elution buffer used for providing the alpha-lactalbumin fraction and the beta-lactoglobulin fraction correspond to at the most 5 times the volume of the chromatographic support, such as at most 4 times the volume of the chromatographic support, e.g. at most 3 times the volume of the chromatographic support, such as at most 2 times the volume of the chromatographic support, e.g. at most 1 times the volume of the chromatographic support.

In the context of the present invention, the terms “column volumes” and “volume of the chromatographic support” are used interchangeable and relates to the volume of the adsorbent present in the chromatographic support that is capable of separating and retaining beta-lactoglobulin from alpha-alpha-lactalbumin, e.g. the adsorbent.

In an embodiment of the present invention, the consumption of elution buffer per kg beta-lactoglobulin fraction, determined as dried beta-lactoglobulin fraction, is preferably less than 250 L/kg dried beta-lactoglobulin fraction, such as less than 200 L/kg dried beta-lactoglobulin fraction, e.g. less than 150 L/kg dried beta-lactoglobulin fraction, such as less than 100 L/kg dried beta-lactoglobulin fraction, e.g. less than 90 L/kg dried beta-lactoglobulin fraction, such as less than 80 L/kg dried beta-lactoglobulin fraction, e.g. less than 75 L/kg dried beta-lactoglobulin fraction, such as less than 70 L/kg dried beta-lactoglobulin fraction, e.g. less than 60 L/kg dried beta-lactoglobulin fraction.

This significant reduction in consumption of elution buffer for providing two fractions according to the present invention, namely the alpha-lactalbumin fraction and the beta-lactoglobulin fraction, is a significant improvement of the technology described in the prior art.

In order to control elution it is possible to use a change in pH, add salts or a combination hereof. In the present invention the beta-lactoglobulin fraction may be provided by changing the pH. Alternatively, in the event several proteins are adsorbed, together with beta-lactoglobulin, to the chromatographic support, selective elution may be used for the sequential elution of the beta-lactoglobulin and the remaining proteins adsorbed to the chromatographic support.

Preferably, the beta-lactoglobulin fraction may be eluted by changing the pH. In a preferred embodiment of the present invention, the pH of the elution buffer may facilitate optimal desorption of beta-lactoglobulin adsorbed to the chromatographic support. In a preferred embodiment of the present invention the elution buffer has a pH above 6.5, e.g. a pH of at least 7.0, such as at least 8.0, e.g. at least 9.5, such as at least 10.5, e.g. at least 11.5, such as at least 12.0. In yet an embodiment of the present invention the elution buffer has a pH value in the range of 7.0-13.0, such as in the range of 8.0-12.5, e.g. in the range of 9.0-12.0, such as in the range of 10.0-11.5, e.g. in the range of 10.5-11.0, preferably in the range of 11.5-12.5.

In an embodiment of the present invention the elution buffer may comprise sodium hydroxide, potassium hydroxide, calcium hydroxide, ammonium hydroxide, potassium phosphate, sodium phosphate, sodium citrate, sodium acetate, sodium carbonate or any combinations hereof. Preferably, the elution buffer comprises sodium hydroxide, potassium hydroxide, calcium hydroxide, ammonium hydroxide or any combination hereof is preferred.

### The Beta-Lacto-Globulin Fraction

The retentate fraction obtained may preferably comprise the beta-lactoglobulin fraction and depending on the type of whey material contacted with the chromatographic material and/or the composition of the elution buffer, the beta-lactoglobulin fraction may comprise other components.

In an embodiment of the present invention the beta-lactoglobulin fraction obtained in step (v) has a conductivity at 20° C. below 50 mS/cm, such as below 40 mS/cm, e.g. below 30 mS/cm, such as below 25 mS/cm, e.g. below 20 mS/cm, such as below 15 mS/cm, e.g. below 10 mS/cm, such as below 8 mS/cm, e.g. below 5 mS/cm, such as below 3 mS/cm, e.g. below 2 mS/cm, such as below 1 mS/cm, e.g. below 0.5 mS/cm. Preferably, the conductivity is determined directly on the beta-lactoglobulin fraction obtained from step (v).

In another embodiment of the present invention, the beta-lactoglobulin fraction has a pH value above 4.5, e.g. at least 5.5, such at least 6.5, e.g. a pH of at least 7.0, such as at least 8.0, e.g. at least 9.5, such as at least 10.5, e.g. at least 11.5, such as at least 12.0.

The inventors of the present invention surprisingly found that one of the advantages of the present invention is the high amount of beta-lactoglobulin recovered from the whey material. In an embodiment of the present invention more than 80% of the beta-lactoglobulin present in the whey material is in the beta-lactoglobulin fraction, e.g. more than 90% of the beta-lactoglobulin present in the whey material is in the beta-lactoglobulin fraction, such as at least 91%, e.g. at least 92%, such as at least 93%, e.g. at least 94%, such as at least 95%, e.g. at least 96%, such as at least 97%, e.g. at least 98%, such as at least 99%, e.g. at least 99.5%. This high recovery of the beta-lactoglobulin fraction may preferably be obtained from a single contact between the whey material and the chromatographic support.

In the present context, the term “single contact” relates to contacting the whey material only one time with the chromatographic support and without re-cycling the permeate fraction or the retentate fraction to the chromatographic support to improve the separation.

Depending on the type of whey material used or the elution buffer used, e.g. if selective elution is used, the purity of the beta-lactoglobulin fraction may be further improved. In an embodiment of the present invention the amount of beta-lactoglobulin in the beta-lactoglobulin fraction is a least 75% relative to the total amount of protein in the beta-lactoglobulin fraction, such as at least 80%, e.g. 90%, such as at least 91%, e.g. 92%, such as at least 93%, e.g. at least 94%, such as at least 95%, e.g. at least 96%, such as at least 97%, e.g. at least 98%, such as at least 99%, e.g. at least 99.5%.

The beta-lactoglobulin fraction may preferably comprise less than 25% (w/w) of non-beta-lactoglobulin proteins relative to the total amount of protein in the beta-lactoglobulin fraction, preferably, less than 15% (w/w) of non-beta-lactoglobulin proteins relative to the total amount of protein in the beta-lactoglobulin fraction, more preferably, less than 10%, even more preferably, less than 5%, even more preferably, less than 2%, even more preferably, less than 1%, even more preferably, less than 0.5%, even more preferably, less than 0.1%, even more preferably, less than 0.05%. Preferably, the non-beta-lactoglobulin proteins comprises one or more proteins selected from the group consisting of alpha-lactalbumin, immunoglobulin G, serum albumin, lactoferrin, lactoperoxidase and glycomacropeptide.

In a further embodiment of the present invention the beta-lactoglobulin fraction comprises less than 15% alpha-lactalbumin relative to the total amount of protein in the beta-lactoglobulin fraction, preferably, less than 10%, more preferably, less than 8%, even more preferably, less than 5%, even more preferably, less than 3%, even more preferably, less than 1%.

In yet an embodiment of the present invention the beta-lactoglobulin fraction comprises less than 10% immunoglobulin G relative to the total amount of protein in the beta-lactoglobulin fraction, more preferably, less than 5%, even more preferably, less than 3%, even more preferably, less than 2%, even more preferably, less than 1%, even more preferably, less than 0.5%, even more preferably, less than 0.1%, even more preferably, less than 0.05%.

In the event the whey protein depleted or substantially depleted from the whey is not immunoglobulin G the beta-lactoglobulin fraction may comprise higher amount of immunoglobulin G such as less than 60% immunoglobulin relative to the total amount of protein in the beta-lactoglobulin fraction, such as less than 50%, e.g. less than 40%, e.g. less than 30%, such as less than 20%, e.g. less than 10%, such as less than 5%.

In an even further embodiment of the present invention the beta-lactoglobulin fraction comprises less than 5% glycomacropeptide relative to the total amount of protein in the beta-lactoglobulin fraction, more preferably, less than 4%, even more preferably, less than 3%, even more preferably, less than 2%, even more preferably, less than 1%, even more preferably, less than 0.5%, even more preferably, less than 0.1%, even more preferably, less than 0.05%.

In yet an embodiment of the present invention, the beta-lactoglobulin fraction may comprises minerals. Preferably, the mineral may be selected from the group consisting of calcium, phosphorus, iodine, magnesium, zinc and potassium. Even more preferably, the mineral may be calcium and a second mineral selected from the group consisting of phosphorus, iodine, magnesium, zinc and potassium.

Even though the beta-lactoglobulin fraction may comprise minerals, the main part of the minerals may be in the run through fraction and ends up in the alpha-lactalbumin fraction. Hence, the beta-lactoglobulin fraction may comprises less that 20% minerals relative to the total amount of minerals in the whey material, such as less than 15% minerals, e.g. less than 10% minerals, such as less than 5% minerals, e.g. less than 1% minerals.

In an embodiment of the present invention, the beta-lactoglobulin fraction obtained may be subjected to a second concentration step. Such second concentration step may include ultrafiltration, nanofiltration, microfiltration, centrifugation or any combination hereof. The second concentration step may result in a second concentrated retentate fraction comprising the beta-lactoglobulin fraction and a second concentrated permeate fraction mainly comprising water. In an embodiment of the present invention the water obtained in the second concentrated permeate fraction may preferably be reused in the method according the present invention.

In an embodiment of the present invention, the beta-lactoglobulin fraction is a liquid, a concentrate or a powder.

### Further Embodiments

In most of the prior art, denaturation of whey proteins are not considered an issue, and process conditions are implemented that may jeopardise the functionality of the native whey proteins.

The present invention may benefit from the very gentle handling of the whey proteins and it may preferably be desired that the native functionality/functionalities of the alpha-lactalbumin fraction and/or the beta-lactoglobulin fraction may be maintained, more preferably, the native functionality/functionalities of the alpha-lactalbumin fraction and the beta-lactoglobulin fraction may be maintained.

Different conditions may cause denaturation of whey proteins, and some proteins in the whey material may be more sensitive than others. Examples of conditions that may cause denaturation may be exposure to pH values below 3 and above 12; high salt concentrations; heat; and chemicals.

Thus in order to avoid denaturation of whey proteins the milk has preferably not been subjected to pasteurisation. Likewise the whey material has preferably not been subjected to pasteurisation.

Even though high temperatures should be avoided in order not to risk denature of the whey proteins, the method according to the present invention may advantageously be conducted at temperatures above ambient temperature. In a further embodiment of the present invention at least one of the steps (ii) to (v) may be performed at a temperature above 25° C., such as above 27° C., e.g. above 30° C., such as above 35° C., e.g. above 40° C., such as above 45° C., e.g. about 50° C., such as in the range of 25-80° C., e.g. in the range of 30-70° C., such as in the range of 35-65° C., e.g. in the range of 40-60° C., such as in the range of 45-55° C.

The purity, yield and recovery of alpha-lactalbumin fraction and beta-lactoglobulin fraction obtained from the whey material as described in the present invention may be provided from a single cycle of the whey material through the chromatographic support.

In the present context the term “single cycle” relates to only one time contact between the chromatographic support and the whey material. The permeate fraction or the retentate fraction are not re-cycled to the chromatographic support in order to provide further separation of the alpha-lactalbumin fraction and the beta-lactoglobulin fraction.

In a preferred embodiment of the present invention the alpha-lactalbumin fraction according to the present invention and/or the beta-lactoglobulin fraction according to the present invention may be used as an ingredient in a food product, a feed product, beverage product, a cosmetic product, a pharmaceutical product, or a food supplement.

Alpha-lactalbumin fraction of the present invention may preferably be used in an infant formula.

The beta-lactoglobulin fraction of the present invention may be used in several applications, in particular in several food applications. Preferably, the beta-lactoglobulin fraction of the present invention may be used as a stabilizer in beverages, wherein the beta-lactoglobulin may stabilize other proteins in the beverage that otherwise may precipitate in acidic environments causing the beverage to become unclear and unattractive.

In another embodiment, the beta-lactoglobulin fraction of the present invention may be used at a carrier for vitamins as beta-lactoglobulin comprise binding properties for e.g. vitamins.

In yet an embodiment, the beta-lactoglobulin fraction of the present invention has shown to have strong foaming properties and may preferably be used as a foaming agent, e.g. in substituting egg-white.

In yet an embodiment, the beta-lactoglobulin fraction of the present invention may be used as a sport nutrition, preferably, a sport nutrition in the form of a winegum, a gel, a beverage, a powder, a pill or a syrup to improve recovery, such as muscle recovery, from heavy exercise.

It should be noted that embodiments and features described in the context of one of the aspects of the present invention also apply to the other aspects of the invention.

All patent and non-patent references cited in the present application, are hereby incorporated by reference in their entirety.

### Theoretical Example of the Method of the Present Invention

In the following, a theoretical example of the method according to the present invention is described. In this theoretical example the whey material provided for the separation of alpha-lactoalbumin and beta-lactoglobulin has been depleted, or substantially depleted, from one of more whey proteins. In the following theoretical example of the preferred embodiment of the method of the present invention, immunoglobulin G is initially removed, or substantially removed, from the whey material. The resulting permeate obtained (the first permeate) represent the depleted, or substantially depleted whey material subjected to the separation of alpha-lactalbumin and beta-lactoglobulin.

Definitions and embodiments described earlier in the present patent application also apply here in the theoretical example of the method of the present invention, unless stated otherwise.

Thus, in one aspect the present invention according to the theoretical example, the present invention relates to a method for providing an immunoglobulin G fraction, an alpha-lactalbumin fraction and a beta-lactoglobulin fraction from a whey material obtained from milk, the method comprising the steps of:
(i) Proving the whey material;
(ii) Contacting the whey material with a first chromatographic support allowing immunoglobulin G to be retained by the first chromatographic support;
(iii) Obtaining a first permeate fraction comprising the alpha-lactalbumin fraction and the beta-lactoglobulin fraction;
(iv) Obtaining a first retentate fraction from the first chromatographic support comprising the immunoglobulin G fraction;
(v) Contacting the first permeate fraction with a second chromatographic support allowing beta-lactoglobulin to be retained by the second chromatographic support;
(vi) Obtaining a second permeate fraction from the chromatographic support comprising the alpha-lactoalbumin fraction; and
(vii) Obtaining a second retentate fraction from the chromatographic material comprising the beta-lactoglobulin fraction.

In a preferred embodiment of the present invention the fractionation of the immunoglobulin G fraction from the whey material comprising the alpha-lactalbumin fraction and the beta-lactoglobulin fraction may be performed by selective adsorption of the immunoglobulin G fraction to the first chromatographic support.

In a preferred embodiment of the present invention the fractionation of the alpha-lactalbumin fraction from the beta-lactoglobulin fraction from the first permeate, may be performed by selective adsorption of the beta-lactoglobulin fraction to the second chromatographic support.

In an embodiment of the present invention the selective adsorption results in the separation of an immunoglobulin G fraction from alpha-lactalbumin and beta-lactoglobulin. This separation may be performed by providing a first chromatographic support and/or first process conditions, which favour selective adsorption of immunoglobulin G and allow beta-lactoglobulin and alpha-lactalbumin to pass the first chromatographic support with the first permeate fraction, without being adsorbed.

In a further embodiment of the present invention the selective adsorption results in a separation of the alpha-lactalbumin fraction from the beta-lactoglobulin fraction. This separation may be performed by providing a second chromatographic support and/or second process conditions, which favour selective adsorption of beta-lactoglobulin and allow alpha-lactalbumin to pass the second chromatographic support with the second permeate fraction, without being adsorbed. Some process conditions are already mentioned previously.

In the present context, the term “whey material” relates to the serum material from milk, the part of milk without casein. Whey material according to the present invention may be whey, acidic whey, sweet whey, at least partly fractionated whey (as long as immunoglobulin G, alpha-lactalbumin and beta-lactoglobulin are present in the at least partly fractionated whey), whey protein isolates (WPI) or whey protein concentrates (WPC).

In the present context, the term “at least partly fractionated whey” relates to whey where at least one protein has been removed, or substantially removed. The only requirement for this type of whey is the presence of immunoglobulin G, alpha-lactalbumin and beta-lactoglobulin.

As described above, the present invention teach the use of a first chromatographic support allowing immunoglobulin G to be retained, step (iv) and the use of a second chromatographic support allowing beta-lactoglobulin to be retained, step (vii).

In the present context the term “first chromatographic support” relates to a chromatographic support provided for retaining immunoglobulin G. In the present context, the term “second chromatographic support” relates to a chromatographic support provided for retaining beta-lactoglobulin.

In a preferred embodiment of the present invention, the first chromatographic support may comprise a first adsorbent.

This first adsorbent may be used in a technique selected from the group consisting of ion exchange adsorption, hydrophobic interaction adsorption, affinity adsorption, mixed mode ligand adsorption, metal chelate adsorption, reversed phase adsorption, and any combination hereof.

In a preferred embodiment of the present invention the first adsorbent is used in mixed mode ligand adsorption.

In a preferred embodiment of the present invention, the second chromatographic support comprises a second adsorbent.

This second adsorbent may be used in a technique selected from the group consisting of ion exchange adsorption, hydrophobic interaction adsorption, affinity adsorption, mixed mode ligand adsorption, metal chelate adsorption, reversed phase adsorption, and any combination hereof.

In a preferred embodiment of the present invention, the adsorbent may be used in ion exchange adsorption, preferably, in cation exchange adsorption.

In an embodiment of the present invention, the second adsorbent may be used in ion exchange adsorption, preferably, in anion exchange adsorption.

In a preferred embodiment, the equilibration liquid used in anion exchange adsorption and may be a liquid having a pH above 6.5, such a pH above 7.0, e.g. a pH above 7.5, such a pH above 8.0, e.g. a pH above 8.5, such a pH above 9.0, such as in the range of pH 7.0-9.0.

In an embodiment of the present invention the equilibration liquid used in anion exchange adsorption may comprise sodium hydroxide, potassium hydroxide, calcium hydroxide, ammonium hydroxide, potassium phosphate, sodium phosphate, sodium citrate, sodium acetate, sodium carbonate or any combinations hereof. Preferably, the elution buffer comprises sodium hydroxide, potassium hydroxide, calcium hydroxide, ammonium hydroxide or any combination hereof is preferred.

In an embodiment of the present invention the adsorbent used in the first chromatographic support has a different mean particle size compared to the adsorbent used in the second chromatographic support. Preferably, the mean particle size of the first chromatographic support is in the range of 160-220 μm, such as in the range of 170-200 μm, e.g. in the range of 175-190 μm, such as about 180 μm. In another embodiment of the present invention the mean particle size of the second chromatographic support is in the range of 120-159 μm, such as in the range of 130-150 μm, e.g. in the range of 135-145 μm, such as about 140 μm.

During operation, the whey material may be contacted with the first adsorbent and immunoglobulin G may be adsorbed or fixated to the first adsorbent. This adsorption may be performed under pressure. Beta-lactoglobulin and alpha-lactalbumin are allowed to pass through the first chromatographic support and the first adsorbent without binding thereto, or substantially without binding thereto. Particulate material and soluble impurities are optionally removed from the first adsorbent during an optional washing.

When contacting the whey material with the first adsorbent, the ratio between the first adsorbent and the whey material may be optimized in order to provide a high capacity of the first adsorbent and to obtain a high purity and/or high recovery of the immunoglobulin G fraction.

In an embodiment of the present invention the loading ratio of immunoglobulin G relative to the first adsorbent is at least 2 mg immunoglobulin G loaded per ml first adsorbent, such as at least 5 mg, e.g. at least 10 mg, such as at least 12 mg, e.g. at least 15 mg, such as at least 20 mg, e.g. at least 25 mg, such as at least 30 mg, e.g. at least 35 mg, such as at least 40 mg, e.g. at least 50 mg, such as at least 75 mg, e.g. at least 100 mg, such as at least 125 mg, e.g. at least 150 mg.

In a further embodiment of the present invention the loading ratio of the whey relative to the first adsorbent is at least 10 mg protein loaded per ml first adsorbent, such as at least 12 mg, e.g. at least 15 mg, such as at least 20 mg, e.g. at least 25 mg, such as at least 30 mg, e.g. at least 35 mg, such as at least 50 mg, e.g. at least 75 mg, such as at least 100 mg, e.g. at least 150 mg, such as at least 175 mg, e.g. at least 200 mg.

When loading the whey material on to the first chromatographic support a first permeate is provided comprising uncaptured and unbound materials, such as beta-lactoglobulin and alpha-lactalbumin.

The first permeate may be contacted with the second adsorbent and beta-lactoglobulin may be adsorbed or fixated to the second adsorbent. This adsorption may be performed under pressure. Alpha-lactalbumin is allowed to pass through the second chromatographic support and the second adsorbent without binding thereto, or substantially without binding thereto. Particulate material and soluble impurities are optionally removed from the second adsorbent during an optional washing.

When contacting the first permeate fraction with the second adsorbent, the ratio between the second adsorbent and the first permeate fraction may be optimized in order to provide a high capacity of the second adsorbent and to obtain a high purity, high yield and/or high recovery of the beta-lactoglobulin fraction and the alpha-lactalbumin fraction.

In an embodiment of the present invention the loading ratio of beta-lactoglobulin relative to the second adsorbent is at least 2 mg beta-lactoglobulin loaded per ml second adsorbent, such as at least 5 mg, e.g. at least 10 mg, such as at least 12 mg, e.g. at least 15 mg, such as at least 20 mg, e.g. at least 25 mg, such as at least 30 mg, e.g. at least 35 mg, such as at least 40 mg, e.g. at least 50 mg, such as at least 75 mg, e.g. at least 100 mg, such as at least 125 mg, e.g. at least 150 mg.

In another embodiment of the present invention the loading ratio of the whey relative to the second adsorbent is at least 10 mg protein loaded per ml second adsorbent, such as at least 12 mg, e.g. at least 15 mg, such as at least 20 mg, e.g. at least 25 mg, such as at least 30 mg, e.g. at least 35 mg, such as at least 50 mg, e.g. at least 75 mg, such as at least 100 mg, e.g. at least 150 mg, such as at least 175 mg, e.g. at least 200 mg.

In order for the adsorbent to act in selective adsorption of immunoglobulin G from beta-lactoglobulin and alpha-lactalbumin the first adsorbent may comprise a first ligand.

In a preferred embodiment of the present invention the first adsorbent comprises one or more first ligands having affinity for immunoglobulin G.

In the present context the term “first ligand” relates to a compound covalently attached to the first adsorbent and which possesses the adsorbing function of immunoglobulin G.

In an embodiment of the present invention the first adsorbent may be used in mixed mode ligand adsorption.

In a further embodiment of the present invention the first ligand comprises an acidic mono- or bicyclic, optionally substituted, aromatic or heteroaromatic moiety.

Preferably, the acidic mono- or bicyclic, optionally substituted, aromatic or heteroaromatic moiety is a benzoic acid or a substituted benzoic acid.

The first ligand may be a low molecular weight compound and in an embodiment of the present invention the ligand may have a molecular weight of at the most 500 Dalton, such as at the most 250 Dalton, e.g. at the most 100 Dalton, e.g. at the most 50 Dalton.

In a preferred embodiment of the present invention the substituted benzoic acid is selected from the group consisting of 2-aminobenzoic acids, 3-aminobenzoic acids, 4-aminobenzoic acids, 2-mercaptobenzoic acids, 2-mercaptonicotinic acid, 4-amino-2-chlorobenzoic acid, 2-amino-5-chlorobenzoic acid, 2-amino-4-chlorobenzoic acid, 4-aminosalicylic acids, 5-aminosalicylic acids, 3,4-diaminobenzoic acids, 3,5-diaminobenzoic acid, 5-aminoisophthalic acid, 4-aminophthalic acid, preferably, the substituted benzoic acid is 4-aminobenzoic acid.

In order to improve capacity, purity and recovery the first ligand concentration may also be important. Hence, in a preferred embodiment of the present invention the first ligand concentration is in the range of 30-300 μmoles per ml sedimented adsorbent, e.g. 50-200 μmoles per ml sedimented first adsorbent, such as 75-175 μmoles per ml sedimented first adsorbent, e.g. 100-160 μmoles per ml sedimented first adsorbent, such as 120-145 μmoles per sedimented first adsorbent.

In order for the second adsorbent to act in selective adsorption of beta-lactoglobulin from alpha-lactalbumin the second adsorbent may comprise a second ligand.

In a preferred embodiment of the present invention the second adsorbent comprise one or more second ligands having affinity for beta-lactoglobulin.

In the present context the term “second ligand” relates to a compound covalently attached to the second adsorbent and which possesses the adsorbing function of beta-lactoglobulin.

The second ligand may be a low molecular weight compound and in an embodiment of the present invention the ligand may have a molecular weight of at the most 500 Dalton, such as at the most 250 Dalton, e.g. at the most 100 Dalton, e.g. at the most 50 Dalton.

In a preferred embodiment of the present invention the second ligand may be negatively charged at pH 6.5 or below, such as at pH 6.0 or below, e.g. at pH 5.5 or below.

In a further embodiment of the present invention the second ligand may be selected from the group consisting of sulfonic acid ligand(s), such as propane sulphonic acid or butane sulphonic acid, and/or carboxylic acid ligand(s), such as chloroacetic acid.

In an embodiment of the present invention the second ligand may be positively charged at pH above 6.5, such a pH above 7.0, e.g. a pH above 7.5, such a pH above 8.0, e.g. a pH above 8.5, such a pH above 9.0, such as in the range of pH 7.0-9.0.

In yet an embodiment of the present invention the second ligand may be selected from the group consisting of Q-anion exchange ligand(s), such as quaternary ammonium anion, or DEAE-anion exchange ligand(s), such as diethylaminoethyl.

In order to improve capacity, purity and recovery the second ligand concentration may also be important. Hence, in a preferred embodiment of the present invention the second ligand concentration may be in the range of 30-300 μmoles per ml sedimented adsorbent, e.g. 50-200 μmoles per ml sedimented second adsorbent, such as 75-175 μmoles per ml sedimented second adsorbent, e.g. 100-160 μmoles per ml sedimented second adsorbent, such as 120-145 μmoles per sedimented second adsorbent.

In an embodiment of the present invention, the first permeate fraction may comprise minerals. In a preferred embodiment of the present invention the first permeate fraction has not been subjected to removal of minerals. In particular the first permeate fraction has not been subjected to removal of calcium.

Preferably, the mineral is selected from the group consisting of calcium, phosphorus, iodine, magnesium, zinc, and potassium. Preferably, the mineral(s) present in the first permeate fraction is/are naturally present in the whey material.

Preferably, the immunoglobulin G fraction may be eluted by subjecting the first chromatographic support to a first elution buffer. In a preferred embodiment of the present invention, the pH of the first elution buffer may facilitate optimal desorption of immunoglobulin G adsorbed to the first chromatographic support. In a preferred embodiment of the present invention the first elution buffer has a pH above 6.5, e.g. a pH of at least 7.0, such as at least 8.0, e.g. at least 9.5, such as at least 10.5, e.g. at least 11.5, such as at least 12.0. In yet an embodiment of the present invention the first elution buffer has a pH value in the range of 7.0-13.0, such as in the range of 8.0-12.5, e.g. in the range of 9.0-12.0, such as in the range of 10.0-11.5, e.g. in the range of 10.5-11.0, preferably in the range of 11.5-12.5.

Preferably the amount of the first elution buffer used for providing the immunoglobulin G fraction correspond to at most 3 times the volume of the first chromatographic support, such as at most 2 times the volume of the first chromatographic support, e.g. at most 1 times the volume of the first chromatographic support, such as at most 0.5 times the volume of the first chromatographic support, e.g. at most 0.25 times the volume of the first chromatographic support.

In an embodiment of the present invention the amount of immunoglobulin G in the immunoglobulin G fraction may be a least 40% relative to the total amount of protein in the immunoglobulin G fraction, such as at least 50%, e.g. 60%, such as at least 70%, e.g. 80%, such as at least 85%, e.g. at least 87%, such as at least 89%, e.g. about 90%.

In a further embodiment of the present invention, the immunoglobulin G fraction comprises less than 60% non-immunoglobulin G proteins relative to the total amount of protein in the immunoglobulin G fraction, more preferably, less than 50%, even more preferably, less than 40%, even more preferably, less than 30%, even more preferably, less than 25%, even more preferably, less than 20%, even more preferably, less than 15%, even more preferably, less than 10%. Preferably, the non-immunoglobulin G proteins comprises one or more proteins selected from the group consisting of alpha-lactalbumin, beta-lactoglobulin, serum albumin, lactoferrin, lactoperoxidase and glycomacropeptide.

In the present context, the term “second permeate fraction” relates to the fraction running through the second chromatographic support when the first permeate fraction is contacted with the second chromatographic support and the beta-lactoglobulin is retained.

Since the alpha-lactalbumin fraction may be similar to the run through fraction obtained from the second chromatographic support as mentioned above the presence of other components in the alpha-lactalbumin fraction may depend on the type of whey material contacted with the chromatographic material.

In an embodiment of the present invention, the alpha-lactalbumin fraction further comprises lactose, vitamins and/or minerals.

The content of whey proteins in the alpha-lactoglobulin fraction has been described earlier in the present patent application and applies also for this preferred embodiment of the method of the invention.

In order to obtain the second retentate fraction from the second chromatographic support comprising the beta-lactoglobulin fraction, the second chromatographic support may be subjected to a second elution buffer. The term “second elution buffer” may relate to a composition capable of changing the conditions of the second chromatographic support from specific adsorption of beta-lactoglobulin to the release and elution of the beta-lactoglobulin fraction.

In a preferred embodiment of the present invention the amount of second elution buffer used for providing the beta-lactoglobulin fraction may correspond to at the most 5 times the volume of the second chromatographic support, such as at most 4 times the volume of the second chromatographic support, e.g. at most 3 times the volume of the second chromatographic support, such as at most 2 times the volume of the second chromatographic support, e.g. at most 1 times the volume of the second chromatographic support.

The content of whey proteins in the beta-lactoglobulin fraction has been described earlier in the present patent application and applies also for this preferred embodiment of the method of the invention.

### EXAMPLES

### Example 1

Isolation of whey proteins from acid whey using Expanded bed chromatography on a pilot scale using 88 l whey material at different pH-values in the pH-range of pH 3.5-6.6 to illustrate how the different whey proteins bind as a function of pH and show a favourable pH-range where separation of alpha-lactalbumin from beta-lactoglobulin using selective adsorption. Beta-lactoglobulin is adsorbed to the chromatographic support and allowing “pure” alpha-lactalbumin to run through the chromatographic support without binding.

### Raw Material

The whey material used in the present example was obtained from raw milk (bovine), non-pasteurized, which was obtained from a local farmer. The cream was removed from the raw milk by centrifugation. The resulting skim milk was pH-adjusted with hydrochloric acid to pH 4.5. The precipitated casein fraction was removed by passing the whey material through a 100 μm filter net to retain the casein curd. The supernatant, the acid whey material was collected and used in the experiment.

Before adsorption the acid whey material was pH adjusted with 1 M hydrochloric acid for pH-values lower than pH 4.5 respectively 1 M NaOH for pH-values higher than pH 4.5. The table shows the different pH-values tested and the conductivity of the whey material at the respective pH-values.

| pH | Conductivity, mS/cm |
|---|---|
| 3.5 | 10.00 |
| 3.8 | 9.78 |
| 4.0 | 9.18 |
| 4.2 | 9.04 |
| 4.2 | 8.40 |
| 4.5 | 8.14 |
| 4.6 | 7.91 |
| 4.8 | 8.58 |
| 5.0 | 9.31 |
| 5.2 | 9.99 |
| 5.4 | 10.75 |
| 6.0 | 11.18 |
| 6.6 | 11.40 |

### Adsorbent

FastLine SP, a strong cation exchanger comprising sulfonic groups, was used.

The adsorbent is based on 5% agarose with 10% tungsten carbide particles incorporated, density of approximately 2.9 g/ml, and a particle size in the range of 40-250 μm. The adsorbent is cross-linked with epichlorohydrine and coupled with 1,4-butanesultone. Ligand concentration: 174 mmol sulfonic groups/L adsorbent.

### Process Parameters

8.8 litre of adsorbent (FastLine SP) is packed in a chromatographic column having a diameter of 15 cm. The bed height in packed mode is 50 cm.

The adsorbent was equilibrated with up to 50 litres 10 mM sodium citrate to reach the desired pH—see table above.

The pH-adjusted acid whey material was loaded on the chromatographic column, 88 litres. Flow rate, gravity: 15 cm/min resulting in a two times bed expansion (to 100 cm expanded bed height).

The adsorbent is washed with 50 litres water.

The proteins are eluted with 40 litres 20 mM NaOH. The eluate is neutralised with 1 M hydrochloric acid.

The experiment is performed at room temperature (20-25° C.)

### Determination of Protein in the Different Fractions

The yield of the different proteins in the eluates was estimated with SDS-PAGE technique. SDS-PAGE gel electrophoresis was performed according to the following general procedure:

25 μL of sample was mixed with 25 μL tris-glycine sample buffer (LC2676, Novex by Life Technologies, USA). The resulting solution was boiled in water for 5 min under non-reducing conditions. 20 μL of the boiled sample was loaded on to a precast SDS-PAGE gel cassette (4-20% tris-glycine gradient gel (1 mm), (EC6025, Novex by Life Technologies, USA). The gel was running for 1 hour at 200 V, 400 mA. The gel was stained with Coomassie blue dye reagent overnight (SimplyBlue™ SafeStain, LC6060).

### Results

The table below shows the relative yield of alpha-lactalbumin in the eluate obtained from the chromatographic column, after the whey material has been added to the chromatographic column. The yield is shown as percentage of the protein present in the raw material and loaded onto the column for beta-lactoglobulin (beta-LG) and alpha-lactalbumin (alpha-LA). The yield is estimated with SDS-PAGE technique:

| pH | Beta-LG | Alpha-LA |
|---|---|---|
| 3.5 | >95% | >95% |
| 3.8 | >90% | >95% |
| 4.0 | >90% | 90% |
| 4.2 | >90% | 80% |
| 4.4 | >90% | 50% |
| 4.5 | >90% | 20% |
| 4.6 | >90% | 10% |
| 4.8 | 85% | <5% |
| 5.0 | 50% | No binding |
| 5.2 | 20% | No binding |
| 5.4 | 10% | No binding |
| 6.0 | No binding | No binding |
| 6.6 | No binding | No binding |

The results show that the separation of alpha-lactalbumin from beta-lactoglobulin is optimal in the pH-range of 4.5-4.8, where the major part of the beta-lactoglobulin binds to the cation exchanger and is subsequently eluted using 40 litres 20 mM NaOH (resulting in 85→90% beta-lactoglobulin in the eluate) and the major part of the alpha-lactalbumin is recovered in the flow through (Resulting in only <5-20% alpha-lactalbumin present in the eluate).

Even this experiment is done with a whey material comprising all the whey proteins (no depletion of whey proteins has been done), similar results are expected when using a whey material depleted in one or more whey proteins.

### Example 2

Separation of alpha-lactalbumin from beta-lactoglobulin using selective adsorption from an acid whey material depleted from immunoglobulin (IgG), lactoferrin (LF) and lactoperoxidase (LP). Beta-lactoglobulin is adsorbed to the chromatographic support and allowing “pure” alpha-lactalbumin to run through the chromatographic support without binding. The experiment is performed at pH 4.5 and pH 4.7.

### Raw Material

The whey material was an acid whey material obtained from raw milk (bovine), non-pasteurized, collected from a local farmer. The cream was removed from the raw milk by centrifugation. The resulting skim milk was pH-adjusted with hydrochloric acid to pH 4.5.

The precipitated casein fraction was removed by passing the whey material through a 100 μm filter net to retain the casein curd. The supernatant, the acid whey material was substantially depleted from lactoferrin, lactoperoxidase, immunoglobulin G and albumin by chromatographic adsorption and used in the experiments.

Before contacting the acid whey material with the adsorbent, the acid whey material was divided in two fractions, one fraction maintaining the pH-value of the acid whey material (pH 4.5) and one fraction where the pH-value was adjusted with 1 M NaOH for providing a pH-value of 4.7.

### Adsorbent

FastLine SP, strong cation exchanger comprising sulfonic groups.

The adsorbent is based on 5% agarose with 10% tungsten carbide particles incorporated, density of approximately 2.9 g/ml, particle size in the range of 40-250 μm. The adsorbent is cross-linked with epichlorohydrine and coupled with 1,4-butanesultone. Ligand concentration: 174 mmol sulfonic groups/L adsorbent.

### Process Parameters

8.8 litres of adsorbent (FastLine SP) is packed in a chromatographic column having a diameter of 15 cm. The bed height in packed mode is 50 cm.

The adsorbent was equilibrated with up to 50 litres 10 mM sodium citrate to reach pH 4.5 and 4.7, respectively, relative to the two experiments to be conducted.

The pH-adjusted acid whey material was loaded on the chromatographic column, 220 litres. Flow rate, gravity: 15 cm/min resulting in a two times bed expansion (to 100 cm expanded bed height).

The adsorbent is washed with 50 litres water.

The beta-lactoglobulin was eluted with 40 litres 20 mM NaOH. The eluate is neutralised with 1 M hydrochloric acid.

The experiment is performed at room temperature (20-25° C.)

### Determination of Aloha-Lactalbumin and Beta-Lactoglobulin in Fractions

Single Radial Immunodiffusion (SRI) was performed in order to quantify the relative yield in percent of alpha-lactalbumin and beta-lactoglobulin in the flow through fractions from the column loaded at pH 4.5 respectively 4.7 as described in Scand. J. Immunol. Vol. 17, Suppl. 10, 41-56, 1983.

The SRI was performed with: Purified immunoglobulin fraction from hyperimmune rabbit serum raised against bovine alpha-lactalbumin produced by UpFront Chromatography A/S (6.5 μl per cm²), purified immunoglobulin fraction from hyperimmune rabbit serum raised against bovine beta-lactoglobulin produced by UpFront Chromatography A/S (15 μl per cm²),

A standard curve was performed with the acid whey solution loaded onto the column in the concentration of 100%, 80%, 60%, 40% and 20% Each of the fractions was read relative to the standard curve.

### Results

The tables below show the relative yield in percent of the raw material loaded onto the column:

Determination of the amount of alpha-lactalbumin and the amount of beta-lactoglobulin in the flow through fraction from the column, for the two load pH-values.

| Load pH | % alpha-lactalbumin | % beta-lactoglobulin |
|---|---|---|
| 4.5 | 87 | 7 |
| 4.7 | 91 | 11 |

Determination of the amount of alpha-lactalbumin and the amount of beta-lactoglobulin in the eluate obtained from the column, for the two load pH-values.

| Load pH | % alpha-lactalbumin | % beta-lactoglobulin |
|---|---|---|
| 4.5 | 13 | 90 |
| 4.7 | 6 | 89 |

It is concluded that in a load ratio of 1:25 (25 L of whey loaded per L adsorbent) at load 25 pH 4.5, 93% of the beta-lactoglobulin is removed from the acid whey material. At pH 4.7 89% of the beta-lactoglobulin is removed. At both pH-values the major fraction of the alpha-lactalbumin is recovered in the flow through fraction obtained from the adsorbent: 87% and 91% at pH 4.5 and pH 4.7, respectively.

### Example 3

Separation of IgG, alpha-lactalbumin and beta-lactoglobulin using selective adsorption from an acid whey stream. Initially IgG is adsorbed on a first chromatographic support and beta-lactoglobulin and alpha-lactalbumin are allowed to run through the first chromatographic support. Following the first chromatographic support beta-lactoglobulin is adsorbed to a second chromatographic support and allowing “pure” alpha-lactalbumin to run through the second chromatographic support without binding. The experiment is performed at pH 4.6.

### Raw Material

The whey material was an acid whey material obtained from raw milk (bovine), non-pasteurized, collected from a local farmer. The cream was removed from the raw milk by centrifugation. The resulting skim milk was pH-adjusted with hydrochloric acid to pH 4.6. The precipitated casein fraction was removed by passing the whey material through a 100 μm filter net to retain the casein curd. The supernatant, the acid whey material was collected and used in the experiments.

### Adsorbent

The adsorbent for the first chromatographic support was a mixed-mode ligand, comprising a 4-aminobenzoic acid.

The adsorbent for the second chromatographic support was FastLine SP, strong cation exchanger comprising sulfonic groups.

Both adsorbents are based on 5% agarose with 10% tungsten carbide particles incorporated, density of approximately 2.9 g/ml, particle size in the range of 40-250 μm. The mixed mode adsorbents and FastLine SP adsorbents are cross-linked with epichlorohydrine and coupled with 4-aminobenzoic acid and 1,4-butanesultone, respectively. Ligand concentration: 40 mmol mixed mode groups/L adsorbent and 174 mmol sulfonic groups/L adsorbent, respectively.

### Process Parameters

13.2 litre of adsorbent (mixed-mode adsorbent) is packed in a first chromatographic column having a diameter of 15 cm. The bed height in packed mode is 75 cm.

The adsorbent was equilibrated with 75 litres 10 mM sodium citrate to reach pH 4.6.

330 litres pH-adjusted acid whey material was loaded on the first chromatographic column. Flow rate, gravity: 15 cm/min resulting in a two times bed expansion (to 150 cm expanded bed height).

The adsorbent is washed with 75 litres water.

The IgG was eluted with 60 litres 20 mM NaOH. The eluate is neutralised with 1 M hydrochloric acid.

13.2 litres of adsorbent (FastLine SP) is packed in a second chromatographic column having a diameter of 15 cm. The bed height in packed mode is 75 cm.

The adsorbent was equilibrated with 75 litres 10 mM sodium citrate to reach pH 4.6.

330 litres of the run-through fraction obtained from the first chromatographic column was loaded, directly on the second chromatographic column. Flow rate, gravity: 15 cm/min resulting in a two times bed expansion (to 100 cm expanded bed height).

The adsorbent is washed with 75 litres water.

The beta-lactoglobulin was eluted with 60 litres 20 mM NaOH. The eluate is neutralised with 1 M hydrochloric acid.

The experiment is performed at room temperature (20-25° C.)

### Determination of Aloha-Lactalbumin and Beta-Lactoglobulin in Fractions

Single Radial Immunodiffusion (SRI) was performed in order to quantify the relative yield in percent of alpha-lactalbumin and beta-lactoglobulin in the elution fraction and the flow through fraction obtained from the first chromatographic material and in the elution fraction and the flow through fraction obtained from the second chromatographic material as described in Scand. J. Immunol. Vol. 17, Suppl. 10, 41-56, 1983.

The SRI was performed with: Purified immunoglobulin fraction from hyperimmune rabbit serum raised against bovine alpha-lactalbumin produced by UpFront Chromatography A/S (6.5 μl per cm²), purified immunoglobulin fraction from hyperimmune rabbit serum raised against bovine beta-lactoglobulin produced by UpFront Chromatography A/S (15 μl per cm²),

A standard curve was performed with the acid whey solution loaded onto the column in the concentration of 100%, 80%, 60%, 40% and 20%. Each of the fractions was read relative to the standard curve.

### Determination of IgG in the Different Fractions

The yield of IgG was estimated with SDS-PAGE technique.

SDS-PAGE gel electrophoresis was performed according to the following general procedure:

25 μL of sample was mixed with 25 μL tris-glycine sample buffer (LC2676, Novex by Life Technologies, USA). The resulting solution was boiled in water for 5 min under non-reducing conditions. 20 μL of the boiled sample was loaded on to a precast SDS-PAGE gel cassette (4-20% tris-glycine gradient gel (1 mm), (EC6025, Novex by Life Technologies, USA). The gel was running for 1 hour at 200 V, 400 mA. The gel was stained with Coomassie blue dye reagent overnight (SimplyBlue™ SafeStain, LC6060).

### Results

The tables below show the relative yield in percent of the raw material loaded onto the respective columns:

Determination of the amounts of IgG; alpha-lactalbumin and beta-lactoglobulin in the flow through fraction from the first column, at a load pH of 4.6.

| Load pH | % alpha-lactalbumin | % beta-lactoglobulin | % IgG |
|---|---|---|---|
| 4.6 | 100 | 99 | 5 |

Determination of the amounts of IgG, alpha-lactalbumin and beta-lactoglobulin in the flow through fraction from the second column, at a load pH of 4.6.

| Load pH | % alpha-lactalbumin | % beta-lactoglobulin | % IgG |
|---|---|---|---|
| 4.6 | 91 | 5 | 5 |

Determination of the amounts of IgG, alpha-lactalbumin and beta-lactoglobulin in the eluates obtained from the first column and from the second column, eluted with 20 mM NaOH.

| Eluate | % alpha-lactalbumin | % beta-lactoglobulin | % IgG |
|---|---|---|---|
| First column | 0 | 1 | 95 |
| Second column | 6 | 92 | 0 |

It is concluded that in a loading ratio of 1:25 (25 L of whey loaded per L adsorbent) at load pH 4.6 it is possibly to provide a substantially pure IgG fraction (substantially free from alpha-lactalbumin and beta-lactoglobulin), a substantially pure alpha-lactalbumin fraction (substantially free from IgG and beta-lactoglobulin) and a substantially pure beta-lactoglobulin fraction (substantially free from IgG and alpha-lactalbumin).

The results show that all alpha-lactalbumin and substantially all beta-lactoglobulin are found in the flow through fraction from the first column, whereas 95% of the IgG from the whey material are retained by the 4-aminobenzoic acid conjugated adsorbent present in the first chromatographic support. The retained IgG may subsequently be eluted from the first chromatographic support resulting an IgG fraction free from alpha-lactalbumin and beta-lactoglobulin.

The flow through fraction obtained from the first chromatographic support is subsequently loaded on to the second column, resulting in a flow through fraction (the alpha-lactalbumin fraction) from the second chromatographic support comprising more than 90% of the alpha-lactalbumin, relative to the amount of alpha-lactalbumin present in the whey material originally applied to the first chromatographic support. The alpha-lactalbumin fraction obtained is substantially free from beta-lactoglobulin and IgG, only small amount of these components (about 5% beta-lactoglobulin and 5% IgG) are found in the alpha-lactalbumin fraction, relative to the total amount of these compounds present in the whey material applied to the first chromatographic support.

Beta-lactoglobulin present in the flow through fraction (or in the whey material) are retained by the SP adsorbent present in the second chromatographic support and eluted from the second chromatographic support resulting in the beta-lactoglobulin fraction. The beta-lactoglobulin fraction have shown to comprise no IgG and substantially no alpha-lactalbumin (only about 6%) relative to the amount present in the whey material.

Hence, the method of the present invention have shown to be highly effective providing high yields, recoveries and purities of IgG, alpha-lactalbumin and beta-lactoglobulin in each of the fractions obtained.

### REFERENCES

WO 92/00799
WO 92/18237
WO 97/17132
WO 00/57982
WO 98/33572
WO 2010/037736
EP 0 538 350

We claim:

1. A method for providing an alpha-lactalbumin fraction and a beta-lactoglobulin fraction from a whey material obtained from milk, the method comprising the steps of:
(i) Providing the whey material;
(ii) Contacting the whey material with a chromatographic support allowing beta-lactoglobulin to be retained by the chromatographic support;
(iii) Obtaining a permeate fraction from the chromatographic support comprising the alpha-lactalbumin fraction;
(iv) Optionally washing the chromatographic support;
(v) Obtaining a retentate fraction from the chromatographic support comprising the beta-lactoglobulin fraction;
wherein the whey material provided in step (i) has been depleted, or substantially depleted from at least one whey protein, such as at least 2 whey proteins, e.g. at least 3 whey proteins, and wherein the fractionation of the alpha-lactalbumin fraction from the beta-lactoglobulin fraction is performed by selective adsorption of the beta-lactoglobulin fraction to the chromatographic support.

2. (canceled)

3. (canceled)

4. A method according to claim 1, wherein the whey material provided in step (i) has been depleted or substantially depleted in immunoglobulin G.

5-29. (canceled)

30. The method according to claim 1, wherein the whey material has not been subjected to pasteurisation.

31-36. (canceled)

37. The method according to claim 1, wherein the chromatographic support comprises an adsorbent.

38. (canceled)

39. The method according to claim 37, wherein the adsorbent is used in a technique selected from the group consisting of ion exchange adsorption, hydrophobic interaction adsorption, affinity adsorption, mixed mode ligand adsorption, metal chelate adsorption, reversed phase adsorption, and any combination hereof.

40-43. (canceled)

44. The method according to claim 1, wherein pH of the whey material is adjusted before contacting the whey material with the chromatographic support in step (ii) to a pH in the range of 4.5-6.5, preferably, in the range of pH 4.5-6.0, even more preferably, in the range of pH 4.6-5.5, even more preferably, in the range of pH 4.7-5.0.

45. The method according to claim 1, wherein the conductivity of the whey material is at least 3.0 mS/cm, such as at least 3.5 mS/cm, e.g. at least 4.0 mS/cm, such as at least 4.5 mS/cm, e.g. at least 5 mS/cm, such as at least 5.5 mS/cm, e.g. at least 6, such as at least 7 mS/cm, e.g. at least 7.5 mS/cm, such as in the range of 4.5-15 mS/cm, such as in the range of 5.0-14 mS/cm, e.g. in the range of 5.5-13 mS/cm, such as in the range of 6.0-12.5 mS/cm, e.g. in the range of 6.5-12 mS/cm, such as in the range of 7.0-11.5 mS/cm, e.g. in the range of 7.5-11 mS/cm, such as in the range of 8.0-10.5 mS/cm, e.g. about 9.0 mS/cm.

46. The method according to claim 1, wherein the retentate fraction comprising the beta-lactoglobulin fraction provided in step (v) is obtained by subjecting the chromatographic support to an elution buffer.

47-51. (canceled)

52. The method according to claim 1, wherein the contacting of the whey material with the chromatographic support in step (ii), the wash in step (iv), the obtaining of the retentate fraction from the chromatographic support comprising the beta-lactoglobulin fraction in step (v) or any combination hereof, preferably all three steps, is performed at a temperature above 25° C., such as above 27° C., e.g. above 30° C., such as above 35° C., e.g. above 40° C., such as above 45° C., e.g. about 50° C., such as in the range of 25-80° C., e.g. in the range of 30-70° C., such as in the range of 35-65° C., e.g. in the range of 40-60° C., such as in the range of 45-55° C.

53. An alpha-lactalbumin fraction comprising alpha-lactalbumin obtainable by a method according to claim 1.

54-56. (canceled)

57. The alpha-lactalbumin fraction according to claim 53, wherein the alpha-lactalbumin fraction further comprises lactose.

58-65. (canceled)

66. A beta-lactoglobulin fraction comprising beta-lactoglobulin obtainable by the method according to claim 1.

67-76. (canceled)
